(19) United States
(12) Patent Application Publication
(10) Pub. No.: US 2019/0276905 A1
(43) Pub. Date: Sep. 12, 2019

(54) YEAST WITH IMPROVED ALCOHOL PRODUCTION

(71) Applicant: DANISCO US INC., Palo Alto, CA (US)

(72) Inventors: Celia Payen, Wilmington, DE (US); Zhixiong Xue, Chadds Ford, PA (US)

(21) Appl. No.: 16/348,604

(22) PCT Filed: Nov. 7, 2017
(86) PCT No.: PCT/US17/60311

§ 371 Date: May 9, 2019

Related U.S. Application Data
(60) Provisional application No. 62/419,786, filed on Nov. 9, 2016.

(51) Int. Cl. C12R 1/865; C12P 7/06; C12P 7/16; C07K 14/395

(52) U.S. Cl. CPC C12R 1/865; C07K 14/395; C12P 7/16; C12P 7/06

(57) ABSTRACT

Described are compositions and methods relating to yeast cells having a genetic mutation that give rise to increased stress tolerance and/or increased alcohol production. Such yeast is well-suited for use in alcohol production to reduce fermentation time and/or increase yields.

### PRIORITY

The present application claim priority to U.S. Provisional Application Ser. No. 62/419,786, filed on Nov. 9, 2016, which is hereby incorporated by reference in its entirety.

### TECHNICAL FIELD

The present strains and methods relate to yeast having a genetic mutation that gives rise to increased stress tolerance and/or increased alcohol production. Such yeast is well-suited for use in alcohol production to reduce fermentation time and/or increase yields.

### BACKGROUND

Many countries make fuel alcohol from fermentable substrates, such as corn starch, sugar cane, cassava, and molasses. According to the Renewable Fuel Association (Washington D.C., United States), 2015 fuel ethanol production was close to 15 billion gallons in the United States, alone.

Butanol is an important industrial chemical and drop-in fuel component with a variety of applications including use as a renewable fuel additive, a feedstock chemical in the plastics industry, and a food-grade extractant in the food and flavor industry. Accordingly, there is a high demand for alcohols such as butanol and isobutanol, as well as for efficient and environmentally-friendly production methods.

In view of the large amount of alcohol produced in the world, even a minor increase in the efficiency of a fermenting organism can result in a tremendous increase in the amount of available alcohol. Accordingly, the need exists for organisms that are more efficient at producing alcohol.

### SUMMARY

Described are methods relating to modified yeast cells with increased stress tolerance and/or capable of increased alcohol production. Aspects and embodiments of the compositions and methods are described in the following, independently-numbered paragraphs.

1. In one aspect, modified yeast cells derived from parental yeast cells are provided, the modified cells comprising a genetic alteration that causes the modified cells to produce a decreased amount of functional Dls1 polypeptide compared to the parental cells, wherein the modified cells produce during fermentation (i) an increased amount of alcohol compared to parental cells at the same fermentation temperature and/or (ii) produce the same amount of alcohol compared to the parental cells at a higher fermentation temperature.

2. In some embodiments of the modified cells of paragraph 1, the genetic alteration comprises a disruption of the YJL065c gene present in the parental cells.

3. In some embodiments of the modified cells of paragraph 2, disruption of the YJL065c gene is the result of deletion of all or part of the YJL065c gene.

4. In some embodiments of the modified cells of paragraph 2, disruption of the YJL065c gene is the result of deletion of a portion of genomic DNA comprising the YJL065c gene.

5. In some embodiments of the modified cells of paragraph 2, disruption of the YJL065c gene is the result of mutagenesis of the YJL065c gene.

6. In some embodiments of the modified cells of any of paragraphs 2-5, disruption of the YJL065c gene is performed in combination with introducing a gene of interest at the genetic locus of the YJL065c gene.

7. In some embodiments of the modified cells of any of paragraphs 1-6, the cells do not produce functional Dls1 polypeptide.

8. In some embodiments of the modified cells of any of paragraphs 1-6, the cells do not produce Dls1 polypeptide.

9. In some embodiments, the modified cells of any of paragraphs 1-8 further comprise an exogenous gene encoding a carbohydrate processing enzyme.

10. In some embodiments, the modified cells of any of paragraphs 1-9 further comprise an alteration in the glycerol pathway and/or the acetyl-CoA pathway.

11. In some embodiments, the modified cells of any of paragraphs 1-10 further comprise an alternative pathway for making ethanol.

12. In some embodiments, the modified cells of any of paragraphs 1-11 further comprise a pathway for making butanol.

13. In some embodiments of the modified cells of any of paragraphs 1-12, the cells are of a Saccharomyces spp.

14. In another aspect, a method for producing a modified yeast cell is provided, comprising: introducing a genetic alteration into a parental yeast cell, which genetic alteration reduces or prevents the production of functional Dls1 polypeptide compared to the parental cells, thereby producing modified cells that produces during fermentation (i) an increased amount of alcohol compared to parental cells at the same fermentation temperature and/or (ii) produce the same amount of alcohol compared to the parental cells at a higher fermentation temperature.

15. In some embodiments of the method of paragraph 14, the genetic alteration comprises disrupting the YJL065c gene in the parental cells by genetic manipulation.

16. In some embodiments of the method of paragraph 14 or 15, the genetic alteration comprises deleting the YJL065c gene in the parental cells using genetic manipulation.

17. In some embodiments of the method of any of paragraphs 14-16, disruption of the YJL065c gene is performed in combination with introducing a gene of interest at the genetic locus of the YJL065c gene.

18. In some embodiments of the method of any of paragraphs 14-17, disruption of the YJL065c gene is performed in combination with making an alteration in the glycerol pathway and/or the acetyl-CoA pathway.

19. In some embodiments of the method of any of paragraphs 14-18, disruption of the YJL065c gene is performed in combination with adding an alternative pathway for making ethanol.

20. In some embodiments of the method of any of paragraphs 14-19, disruption of the YJL065c gene is performed in combination with adding a pathway for making butanol.

21. In some embodiments of the method of any of paragraphs 14-20, disruption of the YJL065c gene is performed in combination with introducing an exogenous gene encoding a carbohydrate processing enzyme.

22. In some embodiments of the method of any of paragraphs 14-21, the modified cell is from a Saccharomyces spp.

23. In some embodiments of the method of any of paragraphs 14-22, the alcohol is ethanol and/or butanol.

24. In another aspect, modified yeast cells produced by the method of any of paragraphs 14-23 are provided.

These and other aspects and embodiments of present modified cells and methods will be apparent from the description, including the accompanying Figures.

### BRIEF DESCRIPTION OF THE DRAWINGS

FIG. 1 is a graph showing the estimated volumetric rate, in grams per Liter per hour (g/L/h) of Strain A and B yeast isobutanologens. Strain B includes the YJL065c gene deletion.

FIG. 2 is a graph showing the instantaneous isobutanol production rate, in grams per Liter per hour (g/L/h) of Strain A and B yeast isobutanologens.

### DETAILED DESCRIPTION

### I. Overview

The present compositions and methods relate to modified yeast cells having increased stress tolerance and/or increased alcohol production compared to their parental cells. When used for alcohol production, the modified cells allow fermentations to be performed at a higher temperature, resulting in a reduction in the amount of time required to produce a given amount of alcohol and/or increased production of alcohol in a given fermentation volume. Either or both of these advantages allow alcohol producers to make more alcohol in less time, thereby increasing the supply of alcohol for world consumption.

### II. Definitions

Prior to describing the present strains and methods in detail, the following terms are defined for clarity. Terms not defined should be accorded their ordinary meanings as used in the relevant art.

As used herein, “alcohol” refer to an organic compound in which a hydroxyl functional group (—OH) is bound to a saturated carbon atom.

As used herein, “butanol” refers to the butanol isomers 1-butanol, 2-butanol, tert-butanol, and/or isobutanol (also known as 2-methyl-1-propanol) either individually or as mixtures thereof.

As used herein, “yeast cells” yeast strains, or simply “yeast” refer to organisms from the phyla Ascomycota and Basidiomycota. An exemplary yeast is budding yeast from the order Saccharomycetales. A particular example of yeast is Saccharomyces spp., including but not limited to S. cerevisiae. Yeast include organisms used for the production of fuel alcohol as well as organisms used for the production of potable alcohol, including specialty and proprietary yeast strains used to make distinctive-tasting beers, wines, and other fermented beverages.

As used herein, the phrase “variant yeast cells,” “modified yeast cells,” or similar phrases (see above), refer to yeast that include genetic modifications and characteristics described herein. Variant/modified yeast do not include naturally occurring yeast.

As used herein, the phrase “substantially free of an activity,” or similar phrases, means that a specified activity is either undetectable in an admixture or present in an amount that would not interfere with the intended purpose of the admixture.

As used herein, the terms “polypeptide” and “protein” (and their respective plural forms) are used interchangeably to refer to polymers of any length comprising amino acid residues linked by peptide bonds. The conventional one-letter or three-letter codes for amino acid residues are used herein and all sequence are presented from an N-terminal to C-terminal direction. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, functionally and/or structurally similar proteins are considered to be “related proteins.” Such proteins can be derived from organisms of different genera and/or species, or even different classes of organisms (e.g., bacteria and fungi). Related proteins also encompass homologs determined by primary sequence analysis, determined by secondary or tertiary structure analysis, or determined by immunological cross-reactivity.

As used herein, the term “homologous protein” refers to a protein that has similar activity and/or structure to a reference protein. It is not intended that homologs necessarily be evolutionarily related. Thus, it is intended that the term encompass the same, similar, or corresponding enzyme(s) (i.e., in terms of structure and function) obtained from different organisms. In some embodiments, it is desirable to identify a homolog that has a quaternary, tertiary and/or primary structure similar to the reference protein. In some embodiments, homologous proteins induce similar immunological response(s) as a reference protein. In some embodiments, homologous proteins are engineered to produce enzymes with desired activity(ies).

The degree of homology between sequences can be determined using any suitable method known in the art (see, e.g., Smith and Waterman (1981) Adv. Appl. Math. 2:482; Needleman and Wunsch (1970) J. Mol. Biol., 48:443; Pearson and Lipman (1988) Proc. Natl. Acad. Sci. USA 85:2444; programs such as GAP, BESTFIT, FASTA, and TFASTA in the Wisconsin Genetics Software Package (Genetics Computer Group, Madison, Wis.); and Devereux et al. (1984) Nucleic Acids Res. 12:387-95).

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle, (Feng and Doolittle (1987) J. Mol. Evol. 35:351-60). The method is similar to that described by Higgins and Sharp ((1989) CABIOS 5:151-53). Useful PILEUP parameters including a default gap weight of 3.00, a default gap length weight of 0.10, and weighted end gaps. Another example of a useful algorithm is the BLAST algorithm, described by Altschul et al. ((1990) J. Mol. Biol. 215:403-10) and Karlin et al. ((1993) Proc. Natl. Acad. Sci. USA 90:5873-87). One particularly useful BLAST program is the WU-BLAST-2 program (see, e.g., Altschul et al. (1996)Meth. Enzymol. 266:460-80). Parameters “W,” “T,” and “X” determine the sensitivity and speed of the alignment. The BLAST program uses as defaults a word-length (W) of 11, the BLOSUM62 scoring matrix (see, e.g., Henikoff and Henikoff (1989) Proc. Natl. Acad. Sci. USA 89:10915) alignments (B) of 50, expectation (E) of 10, M'S, N′-4, and a comparison of both strands.

As used herein, the phrases “substantially similar” and “substantially identical,” in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Percent sequence identity is calculated using CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF.

Another indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, the term “gene” is synonymous with the term “allele” in referring to a nucleic acid that encodes and directs the expression of a protein or RNA. Vegetative forms of filamentous fungi are generally haploid, therefore a single copy of a specified gene (i.e., a single allele) is sufficient to confer a specified phenotype.

As used herein, the terms “wild-type” and “native” are used interchangeably and refer to genes proteins or strains found in nature.

As used herein, the term “protein of interest” refers to a polypeptide that is desired to be expressed in modified yeast. Such a protein can be an enzyme, a substrate-binding protein, a surface-active protein, a structural protein, a selectable marker, or the like, and can be expressed at high levels. The protein of interest is encoded by a modified endogenous gene or a heterologous gene (i.e., gene of interest”) relative to the parental strain. The protein of interest can be expressed intracellularly or as a secreted protein.

As used herein, “deletion of a gene,” refers to its removal from the genome of a host cell. Where a gene includes control elements (e.g., enhancer elements) that are not located immediately adjacent to the coding sequence of a gene, deletion of a gene refers to the deletion of the coding sequence, and optionally adjacent enhancer elements, including but not limited to, for example, promoter and/or terminator sequences, but does not require the deletion of non-adjacent control elements.

As used herein, “disruption of a gene” refers broadly to any genetic or chemical manipulation, i.e., mutation, that substantially prevents a cell from producing a function gene product, e.g., a protein, in a host cell. Exemplary methods of disruption include complete or partial deletion of any portion of a gene, including a polypeptide-coding sequence, a promoter, an enhancer, or another regulatory element, or mutagenesis of the same, where mutagenesis encompasses substitutions, insertions, deletions, inversions, and combinations and variations, thereof, any of which mutations substantially prevent the production of a function gene product. A gene can also be disrupted using RNAi, antisense, or any other method that abolishes gene expression. A gene can be disrupted by deletion or genetic manipulation of non-adjacent control elements.

As used herein, the terms “genetic manipulation” and “genetic alteration” are used interchangeably and refer to the alteration/change of a nucleic acid sequence. The alteration can include but is not limited to a substitution, deletion, insertion or chemical modification of at least one nucleic acid in the nucleic acid sequence.

As used herein, a “primarily genetic determinant” refers to a gene, or genetic manipulation thereof, that is necessary and sufficient to confer a specified phenotype in the absence of other genes, or genetic manipulations, thereof. However, that a particular gene is necessary and sufficient to confer a specified phenotype does not exclude the possibility that additional effects to the phenotype can be achieved by further genetic manipulations.

As used herein, a “functional polypeptide/protein” is a protein that possesses an activity, such as an enzymatic activity, a binding activity, a surface-active property, or the like, and which has not been mutagenized, truncated, or otherwise modified to abolish or reduce that activity. Functional polypeptides can be thermostable or thermolabile, as specified.

As used herein, “a functional gene” is a gene capable of being used by cellular components to produce an active gene product, typically a protein. Functional genes are the antithesis of disrupted genes, which are modified such that they cannot be used by cellular components to produce an active gene product, or have a reduced ability to be used by cellular components to produce an active gene product.

As used herein, yeast cells have been “modified to prevent the production of a specified protein” if they have been genetically or chemically altered to prevent the production of a functional protein/polypeptide that exhibits an activity characteristic of the wild-type protein. Such modifications include, but are not limited to, deletion or disruption of the gene encoding the protein (as described, herein), modification of the gene such that the encoded polypeptide lacks the aforementioned activity, modification of the gene to affect post-translational processing or stability, and combinations, thereof.

As used herein, “attenuation of a pathway” or “attenuation of the flux through a pathway” i.e., a biochemical pathway, refers broadly to any genetic or chemical manipulation that reduces or completely stops the flux of biochemical substrates or intermediates through a metabolic pathway. Attenuation of a pathway may be achieved by a variety of well-known methods. Such methods include but are not limited to: complete or partial deletion of one or more genes, replacing wild-type alleles of these genes with mutant forms encoding enzymes with reduced catalytic activity or increased Km values, modifying the promoters or other regulatory elements that control the expression of one or more genes, engineering the enzymes or the mRNA encoding these enzymes for a decreased stability, misdirecting enzymes to cellular compartments where they are less likely to interact with substrate and intermediates, the use of interfering RNA, and the like.

As used herein, “aerobic fermentation” refers to growth in the presence of oxygen.

As used herein, “anaerobic fermentation” refers to growth in the absence of oxygen.

As used herein, the singular articles “a,” “an,” and “the” encompass the plural referents unless the context clearly dictates otherwise. All references cited herein are hereby incorporated by reference in their entirety. The following abbreviations/acronyms have the following meanings unless otherwise specified:
° C. degrees Centigrade
AA α-amylase
bp base pairs
DNA deoxyribonucleic acid
DP degree of polymerization
ds or DS dry solids
EtOH ethanol
g or gm gram
g/L grams per liter
GA glucoamylase
GAU/g ds glucoamylase units per gram dry solids
H₂O water
HPLC high performance liquid chromatography
hr or h hour
kg kilogram
M molar
mg milligram
mL or ml milliliter
ml/min milliliter per minute
mM millimolar
N normal
nm nanometer
PCR polymerase chain reaction
ppm parts per million
SAPU/g ds protease units per gram dry solids
SSCU/g ds fungal alpha-amylase units per gram dry solids
Δ relating to a deletion
μg microgram
μL and μl microliter
μM micromolar

### III. Modified Yeast Cells Having Reduced or Eliminated Dls1 Activity

In one aspect, modified yeast cells are provided, the modified yeast having a genetic alteration that causes the cells of the modified strain to produce a decreased amount of functional Dls1 polypeptide (alternatively called Dls1p or YJL065c polypeptide) compared to the corresponding parental cells. Dls1 is a 167-amino acid polypeptide subunit of the ISW2 yeast chromatin accessibility complex (yCHRAC), which contains Isw2, Itc1, Dpb3-like subunit (Dls1), and Dpb4 (see, e.g., Peterson, C. L. (1996) Curr. Opin. Genet. Dev. 6:171-75 and Winston, F. and Carlson, M. (1992) Trends Genet. 8:387-91).

Applicants have discovered that yeast having a genetic alteration that affects Dls1 function exhibit increased robustness in an alcohol fermentation process, allowing higher-temperature, and potentially shorter, fermentations. Shorter fermentation times allow alcohol production facilities to run more fermentation in a given period of time, increasing productivity. Shorter fermentation times and higher fermentation temperatures also reduce the risk of contamination during fermentation and, depending on ambient conditions, reduce the need to cool the fermentation reaction to maintain the viability of the yeast. The modified yeast cells also produce increased amounts of alcohol at an elevated fermentation temperature compared to the parental cells. Increased alcohol production is obviously desirable as it improves the output of an alcohol production facility and represents better carbon utilization from starting plant materials. Without being limited to a theory, it is believed that reducing or elimination the amount of functional Dls1 in yeast cells results in alteration of the function of the ISW2/yCHRAC affecting environmental stress response genes linked to thermotolerance and increased tolerance for alcohol.

The reduction in the amount of functional YJL065c protein can result from disruption of the YJL065c gene present in the parental strain. Because disruption of the YJL065c gene is a primary genetic determinant for conferring the thermotolerant and increased alcohol production phenotypes to the modified cells, in some embodiments the modified cells need only comprise a disrupted YJL065c gene, while all other genes can remain intact. In other embodiments, the modified cells can optionally include additional genetic alterations compared to the parental cells from which they are derived. While such additional genetic alterations are not necessary to confer the described phenotype, they may confer other advantages to the modified cells.

Disruption of the YJL065c gene can be performed using any suitable methods that substantially prevent expression of a function YJL065c gene product, i.e., Dls1. Exemplary methods of disruption as are known to one of skill in the art include but are not limited to: complete or partial deletion of the YJL065c gene, including complete or partial deletion of, e.g., the Dls1-coding sequence, the promoter, the terminator, an enhancer, or another regulatory element; and complete or partial deletion of a portion of the chromosome that includes any portion of the YJL065c gene. Particular methods of disrupting the YJL065c gene include making nucleotide substitutions or insertions in any portion of the YJL065c gene, e.g., the Dls1-coding sequence, the promoter, the terminator, an enhancer, or another regulatory element. Preferably, deletions, insertions, and/or substitutions (collectively referred to as mutations) are made by genetic manipulation using sequence-specific molecular biology techniques, as opposed to by chemical mutagenesis, which is generally not targeted to specific nucleic acid sequences. Nonetheless, chemical mutagenesis can, in theory, be used to disrupt the YJL065c gene.

Mutations in the YJL065c gene can reduce the efficiency of the YJL065c promoter, reduce the efficiency of a YJL065c enhancer, interfere with the splicing or editing of the YJL065c mRNA, interfere with the translation of the YJL065c mRNA, introduce a stop codon into the YJL065c-coding sequence to prevent the translation of full-length tYJL065c protein, change the coding sequence of the Dls1 protein to produce a less active or inactive protein or reduce Dls1interaction with other nuclear protein components, or DNA, change the coding sequence of the Dls1 protein to produce a less stable protein or target the protein for destruction, cause the Dls1 protein to misfold or be incorrectly modified (e.g., by glycosylation), or interfere with cellular trafficking of the Dls1 protein. In some embodiments, these and other genetic manipulations act to reduce or prevent the expression of a functional Dls1 protein, or reduce or prevent the normal biological activity of Dls1.

In some embodiments, the present modified cells include genetic manipulations that reduce or prevent the expression of a functional Dls1 protein, or reduce or prevent the normal biological activity of Dls1, as well as additional mutations that reduce or prevent the expression of a functional Isw2, Itc1, or Dpb4 proteins or reduce or prevent the normal biological activity of Isw2, Itc1, or Dpb4 proteins. In some embodiments, the present modified cells include genetic manipulations that reduce or prevent the expression of a functional Dls1 protein, or reduce or prevent the normal biological activity of Dls1, while having no additional mutations that reduce or prevent the expression of a functional Isw2, Itc1, or Dpb4 proteins or reduce or prevent the normal biological activity of Isw2, Itc1, or Dpb4 proteins.

In some embodiments, the decrease in the amount of functional Dls1 polypeptide in the modified cells is a decrease of at least 30%, at least 40%, at least 50%, at least 60%, at least 70%, at least 80%, at least 90%, at least 95%, at least 98%, at least 99%, or more, compared to the amount of functional Dls1 polypeptide in parental cells growing under the same conditions. In some embodiments, the reduction of expression of functional Dls1 protein in the modified cells is a reduction of at least 30%, at least 40%, at least 50%, at least 60%, at least 70%, at least 80%, at least 90%, at least 95%, at least 98%, at least 99%, or more, compared to the amount of functional Dls1 polypeptide in parental cells growing under the same conditions.

In some embodiments, the increase in alcohol in the modified cells is an increase of at least 1%, at least 2%, at least 3%, at least 4%, at least 5%, or more, compared to the amount of alcohol produced in parental cells growing under the same conditions.

Preferably, disruption of the YJL065c gene is performed by genetic manipulation using sequence-specific molecular biology techniques, as opposed to chemical mutagenesis, which is generally not targeted to specific nucleic acid sequences. However, chemical mutagenesis is not excluded as a method for making modified yeast cells.

In some embodiments, the parental cell that is modified already includes a gene of interest, such as a gene encoding a selectable marker, carbohydrate-processing enzyme, or other polypeptide. In some embodiments, a gene of introduced is subsequently introduced into the modified cells.

The amino acid sequence of the exemplified S. cerevisiae Dls1 polypeptide is shown, below, as SEQ ID NO: 1:

MNNETSGKET ASAPLCSPKL PVEKVQRIAK NDPEYMDTSD
DAFVATAFAT EFFVQVLTHE SLHRQQQQQQ QQVPPLPDEL
TLSYDDISAA IVHSSDGHLQ FLNDVIPTTK NLRLLVEENR
VRYTTSVMPP NEVYSAYVVN DTAPKPNIVE IDLDNDEDDD
EDVTDQE

Based on a BLAST search of the NCBI protein database, the relationship between the amino acid sequence of SEQ ID NO: 1 and other known Saccharomyces spp. Dls1 polypeptides is as shown in Table 1:

TABLE 1
SEQ ID NO: 1 compared to other S. cerevisiae Dls1 polypeptides

| | | | GenBank | SEQ |
|---|---|---|---|---|
| | | % | Accession | ID |
| Description | E value | Identity | No. | NO |
| Dls1p [S. cerevisiae | 1.00E−118 | 100% | NP_012470.1 | 1 |
| S288c] | | | | |
| Dls1p [S. cerevisiae | 3.00E−118 | 99% | EGA86281.1 | 2 |
| VL3] | | | | |
| Dls1p [S. cerevisiae | 5.00E−118 | 99% | AJR74354.1 | 3 |
| YJM1549] | | | | |
| Dls1p [S. cerevisiae | 5.00E−118 | 99% | AJR54899.1 | 4 |
| YJM689] | | | | |
| Dls1p [S. cerevisiae | 6.00E−118 | 99% | AJR53909.1 | 5 |
| YJM681] | | | | |
| Dls1p [S. cerevisiae | 7.00E−118 | 99% | AJR60115.1 | 6 |
| YJM195] | | | | |
| Dls1p [S. cerevisiae | 8.00E−118 | 99% | EGA61649.1 | 7 |
| FostersO] | | | | |
| Dls1p [S. cerevisiae | 9.00E−118 | 99% | AJR64933.1 | 8 |
| YJM555] | | | | |
| Dls1p [S. cerevisiae | 9.00E−118 | 99% | AJV39999.1 | 9 |
| YJM1326] | | | | |
| Dls1p [S. cerevisiae | 2.00E−117 | 99% | AJV41947.1 | 10 |
| YJM1355] | | | | |
| Dls1p [S. cerevisiae | 4.00E−117 | 99% | AJR61100.1 | 11 |
| YJM270] | | | | |
| Dls1p [S. cerevisiae | 5.00E−117 | 99% | AJR64049.1 | 12 |
| YJM470] | | | | |
| DLS1-like protein [S. | 5.00E−77 | 75% | EJT44794.1 | 13 |
| kudriavzevii IFO 1802] | | | | |
| DLS1-like protein | 3.00E−75 | 80% | KOG98540.1 | 14 |
| [S. eubayanus] | | | | |
| Dls1p [S. cerevisiae × | 1.00E−73 | 73% | EHN01737.1 | 15 |
| S. kudriavzevii VIN7] | | | | |
| Dls1p [S. arboricola | 1.00E−68 | 76% | EJS43162.1 | 16 |
| H-6] | | | | |

The amino acid sequence of the Dls1p polypeptide from S. cerevisiae S288c is identical to SEQ ID NO: 1. The amino acid sequence of the Dls1 polypeptides from Table 1 are shown, below:

Dls1p [S. cerevisiae VL3] (SEQ ID NO: 2):
MNNETSGKET ASAPLCSPKL PVEKVQRIAK NDPEYMDTSD
DAFVATAFAT EFFVQVLTHE SLHRQQQQQQ QQVPPLPDEL
TLSYDDISAA IVHSSDGHLQ FLNDVIPTTK NLRLLVEEN
RVRYTTSVMPP NEVYTAYVVN DTAPKPNIVE IDLDNDEDDD
EDVTDQE
Dls1p [S. cerevisiae YJM1549]
(SEQ ID NO: 3):
MNNETSGKET ASAPLCSPKL PVEKVQRIAK NDPEYMDTSD
DAFVATAFAT EFFVQVLTHE SLHRQQQQQQ QQVPPLPDEL
TLSYDDISAA IVHSSDGHLQ FLNDVIPTTK NLRLLVEENR
VRYTTSVMPP NEVYSAYVVN NTAPKPNIVE IDLDNDEDDD
EDVTDQE
Dls1p [S. cerevisiae YJM689]
(SEQ ID NO: 4):
MNNETSGKET ASAPLCSPKL PVEKVQRIAK NDPEYMDTSD
DAFVATAFAT EFFVQVLTHE SLHRQQQQQQ QQVPPLPDEL
TLSYDDISAA IVHSSDGHLQ FLNDVIPTTK NLRLLVEENR
VRYTTSVIPP NEVYSAYVVN DTAPKPNIVE IDLDNDEDDD
EDVTDQE
Dls1p [S. cerevisiae YJM681]
(SEQ ID NO: 5):
MNNETSGKET ASAPLCSPKL PVEKVQRIAK NDPEYMDTSD
DAFVATAFAT EFFVQVLTHE SLHRQQQQQQ QQVPPLPDEL
TLSYDDISAA IVHSSDGHLQ FLNDVIPTTK NLRLLVEENR
VRYTTSVMPP NEVYSAYVVN DTAPKPNIVE IDLDNDEDDD
DDVTDQE
Dls1p [S. cerevisiae YJM195]
(SEQ ID NO: 6):
MNNETSGKET ASAPLCSPKL PVEKVQRIAK NDPEYMDISD
DAFVATAFAT EFFVQVLTHE SLHRQQQQQQ QQVPPLPDEL
TLSYDDISAA IVHSSDGHLQ FLNDVIPTTK NLRLLVEENR
VRYTTSVMPP NEVYSAYVVN DTAPKPNIVE IDLDNDEDDD
EDVTDQE
Dls1p [S. cerevisiae FostersO]
(SEQ ID NO: 7):
MNNETSGKET ASAPLCSPKL PVEKVQRIAK NDPEYMDTSD
BAFVATAFAT EFFVQVLTHE SLHRQQQQQQ QQXPPLPDEL
TLSYDDISAA IVHSSDGHLQ FLNDVIPTTK NLRLLVEENR
VRYTTSVMPP NEVYSAYVVN DTAPKPNIVE IDLDNDEDDD
EDVTDQE
Dls1p [S. cerevisiae YJM555]
(SEQ ID NO: 8):
MNNETSGKET ASAPLCSPKL PVEKVQRIAK NDPEYMDTSD
DAFVATAFAT EFFVQVLTHE SLHRQQQQQQ QQVPALPDEL
TLSYDDISAA IVHSSDGHLQ FLNDVIPTTK NLRLLVEENR
VRYTTSVMPP NEVYSAYVVN DTAPKPNIVE IDLDNDEDDD
EDVTDQE
Dls1p [S. cerevisiae YJM1326]
(SEQ ID NO: 9):
MNNETSGKET ASAPLCSPKL PVEKVQRIAK NDPEYMDTSD
DAFVATAFAT EFFVQVLTHE SLHRQQQQQQ QQVPPLSDEL
TLSYDDISAA IVHSSDGHLQ FLNDVIPTTK NLRLLVEENR
VRYTTSVMPP NEVYSAYVVN DTAPKPNIVE IDLDNDEDDD
EDVTDQE
Dls1p [S. cerevisiae YJM1355]
(SEQ ID NO: 10):
MNNETSGKET ASAPLCSPKL PVEKVQRIAK NDPEYMDTSD
DAFVATAFAT EFFVQVLTHE SLHRQQQQPQ QQVPPLPDEL
TLSYDDISAA IVHSSDGHLQ FLNDVIPTTK NLRLLVEENR
VRYTTSVMPP NEVYSAYVVN DTAPKPNIVE IDLDNDEDDD
EDVTDQE
Dls1p [S. cerevisiae YJM270]
(SEQ ID NO: 11):
MNNETSGKET ASAPLCSPKL PVEKVQRIAK NDPEYMDTSD
DAFVATAFAT EFFVQVLTHE SLHRQQQQQQ QQVPPLPDEL
TLSYDDISAA IVHSSDGHLQ FLNDVIPTTK NLRLLVEENR
VRYITSVMPP NEVYSAYVVN DTVPKPNIVE IDLDNDEDDD
EDVTDQE
Dls1p [S. cerevisiae YJM470]
(SEQ ID NO: 12):
MNNETSGKET ASAPLCSPKL PVEKVQRIAK NDPEYMDTSD
DAFVATAFAT EFFVQVLTHE SLHRQQQQQQ QQVPALPDEL
TLSYDDISAA IVHSSDGHLQ FLNDVIPTTK NLRLLVEENR
VRYTTSVIPP NEVYSAYVVN DTAPKPNIVE IDLDNDEDDD
EDVTDQE
DLS1-like protein [S. kudriavzevii IFO 1802]
(SEQ ID NO: 13):
MSDDTSRIEA ASPPPYSLQL PVEKVQRIAK NDPEYMDTSD
DAFIATALAT ESFIQVLALE SLQHQVPRQV PHPSDEITLS
YDDISGTIVR SADGHLQFLN DVIPMTKNLR LLVEENRVRY
TTSVMPPNEV YSGCVMNETA SKPDIVEIDL DNDEDEDVTD QE
DLS1-like protein [S. eubayanus]
(SEQ ID NO: 14):
MNENTSSIET GVPPPCSLQL PVEKVQRIAK NDPEYMDTSD
DAFVATAFAT EFFIQVLTHE SLQQQQRGQV PHPSDEITLS
YDDVSATILK STDGHLQFLN DVIPITKNLR LLVEENRVRY
TTSVMPPNEV YSTYVMGETA LKPNIVEIDL DNDEDDDEDV
TDQE
Dls1p [S. cerevisiae x S. kudriavzevii
VIN7] (SEQ ID NO: 15):
MSDDTSRIDA ASPPPYSLPA ACGKVQRIAK NDPEYMDTSD
DAFIATALAT ESFIQVLALE SLQHQVPRQV PHPPDEITLS
YDDISGTIVR SADGHLQFLN DVIPMTKNLR LLVEENRVRY
TTSVMPPNEV YSGCVMNETA SKPDIVEIDL DNDEDEDVTD QE
Dls1p [S. arboricola H-6] (SEQ ID NO: 16):
MENDANGTET VSPPPHSPQL PVEKVQRIAK NDPEYMDTSD
DAFVATAFAA QFFIQLLTHE SLQQQQQRHH QILHPSDEIT
LSYDDISATI LRSTDGHLQF LNDVIPLTKN LRLLVEENRV
RYTTSVVPPN EVYSAYMMNE TGVKPNIIEI DLDNDEDDDE
DVTDQE

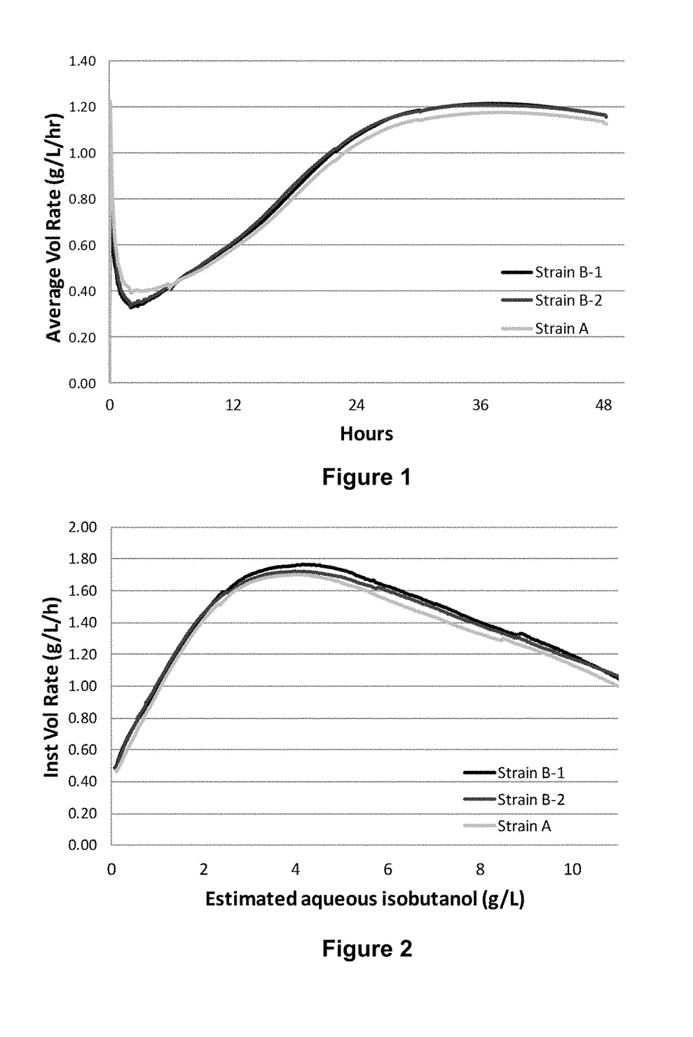

As shown in the sequence alignment in FIG. 1 (performed using Clustal W with default parameters) the amino acid SEQ ID NO: 1 is also 99.4% identical to the Dls1/YJL065c polypeptide mentioned in McIlwain, S. J. et al. ((2016) G3 (Bethesda) 6:1757-66; see Table S3 available on the G3 journal website). The amino acid sequence of KZV10208 is shown, below (SEQ ID NO: 17):

MNNETSGKET ASAPLCSPKL PVEKVQRIAK NDPEYMDTSD
DAFVATAFAT EFFVQVLTHE SLHRQQQQQQ QQVPPLPDEL
TLSYDDISAA IVHSSDGHLQ FLNDVIPTTK NLRLLVEENR
VRYTTSVIPP NEVYSAYVVN DTAPKPNIVE IDLDNDEDDD
EDVTDQE

It is worth noting that McIlwain, S. J. et al. did not identify the Dls1/YJL065c polypeptide or the YJL065c gene as being associated with stress tolerance (including thermotolerance) or alcohol production.

Based on such BLAST and Clustal W data, it is apparent that the exemplified S. cerevisiae Dls1 polypeptide (SEQID NO: 1) share a very high degree of sequence identity to other known S. cerevisiae Dls1 polypeptides, as well as Dls1 polypeptides from other Saccharomyces spp. The present compositions and methods, are therefore, fully expected to be applicable to yeast cells containing such structurally similar polypeptides, as well as other related proteins, homologs, and functionally similar polypeptides.

In some embodiments of the present compositions and methods, the amino acid sequence of the Dls1 protein that is altered in production levels has a specified degree of overall amino acid sequence identity to the amino acid sequence of SEQ ID NO: 1, 2, 3, 4, 5, 6, 7, 8, 9, 10, 11, 12, 13, 14, 15, 16, or 17, e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99% identity, to SEQ ID NO: 1, 2, 3, 4, 5, 6, 7, 8, 9, 10, 11, 12, 13, 14, 15, 16, or 17.

In some embodiments of the present compositions and methods, the YJL065c gene that is disrupted encodes a Dls1 protein that has a specified degree of overall amino acid sequence identity to the amino acid sequence of SEQ ID NO: 1, 2, 3, 4, 5, 6, 7, 8, 9, 10, 11, 12, 13, 14, 15, 16, or 17, e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99% identity, to SEQ ID NO: 1, 2, 3, 4, 5, 6, 7, 8, 9, 10, 11, 12, 13, 14, 15, 16, or 17.

The amino acid sequence information provided, herein, readily allows the skilled person to identify a Dls1 protein, and the nucleic acid sequence encoding a Dls1 protein, in any yeast, and to make appropriate disruptions in the YJL065c gene to affect the production of the Dls1 protein.

### IV. Combination of Decreased Dls1 with Additional Mutations that Affect Alcohol Production

In some embodiments, the present modified cells include any number of additional genes of interest encoding proteins of interest in addition to the genetic alteration that causes the cells of the modified strain to produce a decreased amount of functional Dls1 protein compared to the corresponding parental cells.

In particular embodiments of the compositions and methods the artificial alternative pathway for making ethanol is the result of introducing a heterologous phosphoketolase gene and a heterologous phosphotransacetylase gene. An exemplary phosphoketolase can be obtained from Gardnerella vaginalis (UniProt/TrEMBL Accession No.: WP_016786789). An exemplary phosphotransacetylase can be obtained from Lactobacillus plantarum (UniProt/TrEMBL Accession No.: WP_003641060).

The present modified cells may further include mutations that result in attenuation of the native glycerol biosynthesis pathway, which are known to increase alcohol production. Methods for attenuation of the glycerol biosynthesis pathway in yeast are known and include reduction or elimination of endogenous NAD-dependent glycerol 3-phosphate dehydrogenase (GPD) or glycerol phosphate phosphatase activity (GPP), for example by disruption of one or more of the genes GPD1, GPD2, GPP1 and/or GPP2. See, e.g., U.S. Pat. Nos. 9,175,270 (Elke et al.), 8,795,998 (Pronk et al.) and 8,956,851 (Argyros et al.).

The modified yeast may further feature increased acetyl-CoA synthase (also referred to acetyl-CoA ligase) activity (EC 6.2.1.1) to scavenge (i.e., capture) acetate produced by chemical or enzymatic hydrolysis of acetyl-phosphate (or present in the culture medium of the yeast for any other reason) and converts it to Ac-CoA. This avoids the undesirable effect of acetate on the growth of yeast cells and may further contribute to an improvement in alcohol yield. Increasing acetyl-CoA synthase activity may be accomplished by introducing a heterologous acetyl-CoA synthase gene into cells, increasing the expression of an endogenous acetyl-CoA synthase gene and the like. A particularly useful acetyl-CoA synthase for introduction into cells can be obtained from Methanosaeta concilii (UniProt/TrEMBL Accession No.: WP_013718460). Homologs of this enzymes, including enzymes having at least 85%, at least 90%, at least 92%, at least 95%, at least 97%, at least 98% and even at least 99% amino acid sequence identity to the aforementioned acetyl-CoA synthase from Methanosaeta concilii, are also useful in the present compositions and methods.

In some embodiments the present modified cells may further include a heterologous gene encoding a protein with NAD⁺-dependent acetylating acetaldehyde dehydrogenase activity and/or a heterologous gene encoding a pyruvate-formate lyase. The introduction of such genes in combination with attenuation of the glycerol pathway is described, e.g., in U.S. Pat. No. 8,795,998 (Pronk et al.). In some embodiments of the present compositions and methods the yeast expressly lack a heterologous gene(s) encoding an acetylating acetaldehyde dehydrogenase, a pyruvate-formate lyase or both.

In some embodiments, the present modified yeast cells further comprise a butanol biosynthetic pathway. In some embodiments, the butanol biosynthetic pathway is an isobutanol biosynthetic pathway. In some embodiments, the isobutanol biosynthetic pathway comprises a polynucleotide encoding a polypeptide that catalyzes a substrate to product conversion selected from the group consisting of: (a) pyruvate to acetolactate; (b) acetolactate to 2,3-dihydroxyisovalerate; (c) 2,3-dihydroxyisovalerate to 2-ketoisovalerate; (d) 2-ketoisovalerate to isobutyraldehyde; and (e) isobutyraldehyde to isobutanol. In some embodiments, the isobutanol biosynthetic pathway comprises polynucleotides encoding polypeptides having acetolactate synthase, keto acid reductoisomerase, dihydroxy acid dehydratase, ketoisovalerate decarboxylase, and alcohol dehydrogenase activity.

In some embodiments, the modified yeast cells comprising a butanol biosynthetic pathway further comprise a modification in a polynucleotide encoding a polypeptide having pyruvate decarboxylase activity. In some embodiments, the yeast cells comprise a deletion, mutation, and/or substitution in an endogenous polynucleotide encoding a polypeptide having pyruvate decarboxylase activity. In some embodiments, the polypeptide having pyruvate decarboxylase activity is selected from the group consisting of: PDC1, PDC5, PDC6, and combinations thereof. In some embodiments, the yeast cells further comprise a deletion, mutation, and/or substitution in one or more endogenous polynucleotides encoding FRA2, ALD6, ADH1, GPD2, BDH1, and YMR226C.

### V. Combination of Decreased Dls1 with Additional Proteins of Interest

In some embodiments, in addition to a genetic alteration that causes the cells of the modified strain to produce a decreased amount of functional Dls1 protein compared to corresponding parental cells, optionally in combination with other genetic modifications that benefit alcohol production, the present modified yeast cells further include any number of additional genes of interest encoding proteins of interest. Additional genes of interest may be introduced before, during, or after genetic manipulations that result in reduced expression of functional Dls1 protein.

Proteins of interest, include selectable markers, carbohydrate-processing enzymes, and other commercially-relevant polypeptides, including but not limited to an enzyme selected from the group consisting of a dehydrogenase, a transketolase, a phosphoketolase, a transladolase, an epimerase, a phytase, a xylanase, a β-glucanase, a phosphatase, a protease, an α-amylase, a β-amylase, a glucoamylase, a pullulanase, an isoamylase, a cellulase, a trehalase, a lipase, a pectinase, a polyesterase, a cutinase, an oxidase, a transferase, a reductase, a hemicellulase, a mannanase, an esterase, an isomerase, a pectinases, a lactase, a peroxidase and a laccase. Proteins of interest may be secreted, glycosylated, and otherwise-modified.

### VI. Use of the Modified Yeast for Increased Alcohol Production

The present compositions and methods include methods for increasing the efficiency of alcohol production using the modified yeast in fermentation reactions. The methods include performing fermentation at an elevated temperature and, optionally, a shorter period of time, compared to an otherwise equivalent fermentation performed using the parental cells. For example, the fermentation using the modified yeast cells may be performed at 1° C., 2° C., 3° C., 4° C., 5° C., 6° C., or even 7° C., or more, above the temperature used for the fermentation with the parental yeast cells, provided that the modified yeast is capable of making at least the same amount of alcohol at the increased temperature as the parental yeast make at the reference temperature. The higher temperature fermentation may optionally be run for 99%, 97%, 95%, 90%, 85%, 80%, or less, compared to the amount of time required for fermentation using the parental yeast, provided that the modified yeast is capable of making at least the same amount of alcohol at the increased temperature as the parental yeast make at the reference temperature and time.

Alternatively, the methods include performing fermentation at about the same temperature and about the same length of time compared to an otherwise equivalent fermentation performed using the parental cells, wherein the modified yeast cells produce at least 1%, at least 2%, at least 3%, at least 4%, or even at least 5% more alcohol than the parental yeast under equivalent conditions.

The advantages of the modified yeast in terms of performing fermentations at increased temperatures, performing fermentations for shorter period of time, and increasing alcohol yield under conventional fermentation conditions, can be combined to maximize benefit to a particular alcohol production facility.

In some embodiments, solids may be removed from fermentation media prior to fermentation. In some embodiments, in situ production removal (ISPR) may be utilized to remove product alcohol from fermentation as the product alcohol is produced by the microorganism. Processes for removing solids and producing and recovering alcohols from fermentation broth are described in US Patent Application No. 2014/0073820 and US Patent Application No. 2015/0267225.

### VII. Yeast Cells Suitable for Modification

Yeasts are unicellular eukaryotic microorganisms classified as members of the fungus kingdom and include organisms from the phyla Ascomycota and Basidiomycota. Yeast that can be used for alcohol production include, but are not limited to, Saccharomyces spp., including S. cerevisiae, as well as Kluyveromyces, Lachancea and Schizosaccharomyces spp. Numerous yeast strains are commercially available, many of which have been selected or genetically engineered for desired characteristics, such as high alcohol production, rapid growth rate, and the like. Some yeasts have been genetically engineered to produce heterologous enzymes, such as glucoamylase or α-amylase.

### VIII. Substrates and Products

Alcohol production from a number of carbohydrate substrates, including but not limited to corn starch, sugar cane, cassava, and molasses, is well known, as are innumerable variations and improvements to enzymatic and chemical conditions and mechanical processes. The present compositions and methods are believed to be fully compatible with such substrates and conditions.

Alcohol fermentation products include organic compound having a hydroxyl functional group (—OH) is bound to a carbon atom. Exemplary alcohols include but are not limited to methanol, ethanol, n-propanol, isopropanol, n-butanol, isobutanol, n-pentanol, 2-pentanol, isopentanol, and higher alcohols. The most commonly made fuel alcohols are ethanol, and butanol.

These and other aspects and embodiments of the present strains and methods will be apparent to the skilled person in view of the present description. The following examples are intended to further illustrate, but not limit, the strains and methods.

### EXAMPLES

### Example 1. Deletion of YJL065c in Saccharomyces cerevisiae

Genetic screening was performed to identify thermotolerant S. cerevisiae mutants capable of improved growth at elevated temperature (i.e., 37° C. versus 32° C.) and a number of candidate genes were identified and selected for further testing (data not shown). One of the genes selected for further analysis was YJL065c, which encodes Dls1. The amino acid sequence of Dls1 is provided below as SEQ ID NO: 1.

MNNETSGKET ASAPLCSPKL PVEKVQRIAK NDPEYMDTSD
DAFVATAFAT EFFVQVLTHE SLHRQQQQQQ QQVPPLPDEL
TLSYDDISAA IVHSSDGHLQ FLNDVIPTTK NLRLLVEENR
VRYTTSVMPP NEVYSAYVVN DTAPKPNIVE IDLDNDEDDD
EDVTDQE

Using standard yeast molecular biology techniques, the YJL065c gene was disrupted by deleting essentially the entire coding sequence for Dls1, i.e., by deleting the nucleic acid sequence from 4 base-pair before the start codon to 10 base-pairs before the stop codon in both alleles of S. cerevisiae. All procedures were based on the publically available nucleic acid sequence of YJL065c, which is provided below as SEQ ID NO: 18 (5′ to 3′):

ATGAACAACGAGACTAGTGGTAAAGAAACGGCGTCTGCACCTCTGTGTTC
GCCCAAGTTACCTGTAGAAAAAGTGCAGAGAATAGCCAAGAATGATCCAG
AATATATGGACACTTCGGATGACGCATTCGTAGCCACAGCGTTTGCTACA
GAATTCTTCGTCCAGGTGCTGACACATGAGTCCCTACATAGGCAACAGCA
GCAGCAACAACAACAGGTACCGCCGCTCCCAGATGAACTCACGCTGTCGT
ACGATGACATCTCTGCCGCAATTGTGCACTCTTCTGACGGCCATCTGCAG
TTTTTGAATGATGTGATACCAACAACAAAGAATTTGAGGCTTCTAGTGGA
AGAAAACCGAGTTAGATATACTACAAGTGTCATGCCCCCTAATGAAGTTT
ACTCCGCCTATGTGGTGAACGATACGGCTCCGAAGCCCAACATTGTCGAG
ATTGATCTTGATAATGACGAAGACGACGACGAAGACGTTACTGATCAAGA
ATAA

The host yeast used to make the modified yeast cells was commercially available FERMAX™ Gold (Martrex, Inc., Chaska, Minn., USA). Deletion of the YJL065c gene were confirmed by colony PCR. The modified yeast was grown in non-selective media to remove the plasmid conferring Kanamycin resistance used to select transformants, resulting in modified yeast that required no growth supplements compared to the parental yeast.

### Example 2: Ethanol Production by Modified Yeast

Yeast harboring the deletion of the gene YJL065c (i.e., YCP047) were tested for their ability to produce ethanol compared to benchmark yeast (i.e., FERMAX™ Gold, herein “FG,” which are wild-type for the YJL065c gene) in liquefact at 32, 35 and 37° C. and under the temperature ramp conditions shown in Table 1. Liquefact (i.e., corn flour slurry having a dry solid (ds) value of 35% was prepared by adding 600 ppm urea, 0.124 SAPU/g ds FERMGEN™ 2.5× (an acid fungal protease), 0.33 GAU/g ds CS4 (a variant of Trichoderma reesei glucoamylase) and 1.46 SSCU/g ds AKAA (Aspergillus kawachii α-amylase) at pH 4.8.

TABLE 1
Temperature ramp condition

| Time (hour) | Temperature (° C.) |
|---|---|
| 0-10 | 32 |
| 10-12 | 33 |
| 12-15 | 34 |
| 15-17 | 35 |
| 17-22 | 35.5 |
| 22-27 | 34.5 |
| 27-31 | 34 |
| 31-36 | 33.5 |
| 36-41 | 33 |
| 41-55 | 32.5 |
| 55-end | 32 |

50 grams of liquefact was weighted into 250 ml vessels and inoculated with fresh overnight cultures from colonies of the YCP047 strain or FG strain and incubated at different temperatures. A gas monitoring system (ANKOM Technology) was used to record the rate of fermentation based on cumulative pressure following CO₂ production over time. Samples were harvested by centrifugation, filtered through 0.2 μm filters, and analyzed for ethanol, glucose, acetate and glycerol content by HPLC (Agilent Technologies 1200 series) using Bio-Rad Aminex HPX-87H columns at 55° C., with an isocratic flow rate of 0.6 ml/min in 0.01 N H₂SO₄ eluent. A 2.5 μl sample injection volume was used. Calibration standards used for quantification included known amounts of DP4+, DP3, DP2, DP1, glycerol and ethanol. The results of the analyses are shown in Table 2. Ethanol increase is reported with reference to the FG strain.

TABLE 2
Analysis of fermentation
broth following fermentation with YCP047 and FG yeast

| | | | | | | Ethanol |
|---|---|---|---|---|---|---|
| Temperature | | Glucose | Glycerol | Acetate | Ethanol | increase |
| (° C.) | Strain | (g/L) | (g/L) | (g/L) | (g/L) | (%) |
| 32 | FG | 8.2 | 16.9 | 0.5 | 142.8 | N/A |
| 34 | FG | 22.1 | 16.6 | 0.9 | 136.4 | N/A |
| 37 | FG | 65.6 | 15.2 | 1.1 | 117.7 | N/A |
| Ramp | FG | 8.4 | 16.7 | 0.9 | 137.9 | N/A |
| 32 | YCP047 | 0.9 | 16.5 | 0.6 | 146.0 | 2.3 |
| 34 | YCP047 | 10.5 | 16.7 | 0.8 | 141.4 | 3.7 |
| 37 | YCP047 | 58.7 | 15.6 | 1.3 | 121.7 | 3.4 |
| Ramp | YCP047 | 2.1 | 17.1 | 0.9 | 144.2 | 4.6 |

Yeast harboring the deletion of the gene YJL065c produced significantly more ethanol (i.e., up to almost 5%) compared to the reference strain, particularly at elevated temperatures.

### Example 3: Ethanol Production by Modified Yeast Expressing Glucoamylase

Yeast harboring the deletion of the gene YJL065c (i.e., YCP119) and further expressing the aforementioned CS4 variant of Trichoderma reesei glucoamylase were tested for their ability to produce ethanol compared to benchmark yeast (i.e., SYNERXIA™ ADY, herein “SA,” which are wild-type for the YJL065c gene) using the same conditions and procedures as described in the previous Example. Samples analyzed for ethanol, glucose, acetate and glycerol content and the results are shown in Table 3. Ethanol increase is reported with reference to the SA strain.

TABLE 3
Analysis of fermentation broth following
fermentation with YCP119 and SA yeast

| | | | | | | Ethanol |
|---|---|---|---|---|---|---|
| Temperature | | Glucose | Glycerol | Acetate | Ethanol | increase |
| (° C.) | Strain | (g/L) | (g/L) | (g/L) | (g/L) | (%) |
| 32 | SA | 11.4 | 16.1 | 0.4 | 144.4 | N/A |
| 34 | SA | 38.6 | 15.3 | 0.6 | 127.7 | N/A |
| 37 | SA | 74.8 | 10.9 | 0.6 | 118.2 | N/A |
| Ramp | SA | 30.9 | 15.8 | 0.6 | 134.0 | N/A |
| 32 | YCP119 | 4.5 | 16.1 | 0.4 | 148.6 | 2.9 |
| 34 | YCP119 | 25.8 | 16.0 | 0.6 | 134.2 | 5.1 |
| 37 | YCP119 | 67.5 | 11.4 | 0.6 | 122.5 | 3.6 |
| Ramp | YCP119 | 16.9 | 16.0 | 0.6 | 139.9 | 4.4 |

Yeast harboring the deletion of the gene YJL065c, and also expressing GA, produced significantly more ethanol (i.e., in excess of 5%) compared to the reference strain, particularly at elevated temperatures.

### Example 4: Ethanol Production by Modified Yeast Having an Alternative Ethanol Pathway

Yeast harboring the deletion of the gene YJL065c, and further including an alternative pathway to produce ethanol (i.e., by expressing a heterologous phosphoketolase, a heterologous phosphotransacetylase, and an acetylating acetaldehyde dehydrogenase, as described in international patent application WO 2015/148272 (Miasnikov et al.)), were tested for their ability to produce ethanol compared to parental yeast, which included the alternative ethanol pathway but did not have a deletion of gene YJL065c. In this case, the parental yeast is designated “G032” and the modified yeast is designated “G032-AYJL065c”. Assay conditions and procedures were as described in the previous Examples, except that the yeast were tested only under the temperature ramp conditions described above. Samples were again analyzed for ethanol, glucose, acetate, and glycerol content. The results are shown in Table 4.

TABLE 4
Analysis of fermentation broth following fermentation with G032 yeast

| | | | | | | Ethanol |
|---|---|---|---|---|---|---|
| Temperature | | Glucose | Glycerol | Acetate | Ethanol | increase |
| (° C.) | Strain | (g/L) | (g/L) | (g/L) | (g/L) | (%) |
| Ramp | G032 | 21.32 | 12.45 | 2.08 | 141.80 | n/a |
| Ramp | G032- | 11.75 | 12.70 | 2.11 | 146.64 | 3.4 |
| | ΔYJL065c | | | | | |

As before, increased ethanol production was observed in the yeast harboring the deletion of the gene YJL065c.

### Example 5: Ethanol Production by the Modified Yeast in High Dry Solid at 32° C.

Yeast harboring the deletion of the gene YJL065c (i.e., YCP047) was tested for its ability to produce ethanol compared to FG benchmark yeast in liquefact having a dry solid (DS) value of 36.6% at 32° C. Liquefact (i.e., corn flour slurry) was prepared by adding 600 ppm urea, 0.124 SAPU/g ds FERMGEN™ 2.5× (an acid fungal protease), 0.33 GAU/g ds CS4 (a variant of Trichoderma reesei glucoamylase) and 1.46 SSCU/g ds AKAA (Aspergillus kawachii α-amylase) at pH 4.8.

50 grams of liquefact was weighted into 100 ml vessels and inoculated with fresh overnight cultures from colonies of the YCP047 strain or FG strain and incubated at different temperatures. Samples were harvested by centrifugation at 48 and 55 hours, filtered through 0.2 μm filters, and analyzed for ethanol, glucose, acetate and glycerol content by HPLC (Agilent Technologies 1200 series) using Bio-Rad Aminex HPX-87H columns at 55° C., with an isocratic flow rate of 0.6 ml/min in 0.01 N H₂SO₄ eluent. A 2.5 μl sample injection volume was used. Calibration standards used for quantification included known amounts of ethanol. The results of the analyses are shown in Table 5. Ethanol increase is reported with reference to the FG strain in the same condition.

TABLE 5
Analysis of fermentation broth following
fermentation with YCP047 and FG yeast

| | | | Sampling | | Ethanol |
|---|---|---|---|---|---|
| Temperature | DS | | time | Ethanol | increase |
| (° C.) | (%) | Strains | (hrs) | (g/L) | (%) |
| 32 | 34.4 | FG | 55 | 135.4 | n/a |
| 32 | 34.4 | YCP047 | 55 | 137.9 | 1.8 |
| 32 | 35.5 | FG | 55 | 134.9 | n/a |
| 32 | 35.5 | YCP047 | 55 | 136.2 | 1.0 |

Yeast harboring the deletion of the gene YJL065c produced significantly more ethanol (i.e., up to −2%) compared to the reference strain in liquefact having higher value of dry solid at 32° C.

### Example 6: Ethanol Production by the Modified Yeast in High Dry Solid at 34° C.

Yeast harboring the deletion of the gene YJL065c (i.e., YCP047) was tested for its ability to produce ethanol compared to FG benchmark yeast in liquefact having a dry solid (DS) value of 34.4 and 35.5% dry solid at 34° C. Liquefact (i.e., corn flour slurry) was prepared by adding 600 ppm urea, 0.124 SAPU/g ds FERMGEN′ 2.5× (an acid fungal protease), 0.33 GAU/g ds CS4 (a variant of Trichoderma reesei glucoamylase) and 1.46 SSCU/g ds AKAA (Aspergillus kawachii α-amylase) at pH 4.8.

50 grams of liquefact was weighted into 100 ml vessels and inoculated with fresh overnight cultures from colonies of the YCP047 strain or FG strain and incubated at different temperatures. Samples were harvested by centrifugation at 48 and 55 hours, filtered through 0.2 μm filters, and analyzed for ethanol, glucose, acetate and glycerol content by HPLC (Agilent Technologies 1200 series) using Bio-Rad Aminex HPX-87H columns at 55° C., with an isocratic flow rate of 0.6 ml/min in 0.01 N H₂SO₄ eluent. A 2.5 μl sample injection volume was used. Calibration standards used for quantification included known amounts of ethanol. The results of the analyses are shown in Table 5. Ethanol increase is reported with reference to the FG strain in the same condition.

TABLE 6
Analysis of fermentation broth following
fermentation with YCP047 and FG yeast

| | | | Sampling | | Ethanol |
|---|---|---|---|---|---|
| Temperature | DS | | time | Ethanol | increase |
| (° C.) | (%) | Strains | (hrs) | (g/L) | (%) |
| 34 | 34.4 | FG | 55 | 143.91 | n/a |
| 34 | 34.4 | YCP047 | 55 | 146.29 | 1.7 |
| 34 | 35.5 | FG | 55 | 144.65 | n/a |
| 34 | 35.5 | YCP047 | 55 | 146.26 | 1.1 |

Yeast harboring the deletion of the gene YJL065c produced significantly more ethanol (i.e., up to −2%) compared to the reference strain in liquefact having higher value of DS at 34° C.

### Example 7: Butanol Production by Modified Yeast

Methods for the construction of recombinant S. cerevisiae containing a heterologous pathway for the production of isobutanol (i.e., isobutanologens) are described in U.S. Pat. Nos. 9,422,581, 9,169,467, and 8,409,834 and US Patent Application Publication Nos. 2014/0051133 and 2014/0093930, each of which is incorporated by reference in its entirety.

An isobutanologen was engineered to contain a heterologous isobutanol pathway consisting of acetolactate synthase, ketol acid reductoisomerase, dihydroxyacid dehydratase, ketoisovalerate decarboxylase, and alcohol dehydrogenase genes (herein referred to as “Strain A”). A further yeast strain (herein referred to as “Strain B”) was constructed by deletion of gene YJL065c in Strain A as described, above.

Isobutanologen Strains A (a single isolate) and B (isolates 1 and 2) were grown for 48 hours at 32° C. in glass bottles equipped with the ANKOM RF Gas Production System (ANKOM Technology, Macedon N.Y.) using filtered corn mash media and 50% (w/v) corn oil fatty acids as extraction solvent. Glucoamylase enzyme was added to convert starch into glucose. Isobutanol production was estimated by measurement of evolved carbon dioxide using the ANKOM system.

Strain B containing the deletion of gene YJL065c exhibited higher volumetric production rates (FIG. 1) at higher aqueous isobutanol concentrations (FIG. 2) compared to Strain A, which does not contain the gene YJL065c deletion.

What is claimed is:

1. Modified yeast cells derived from parental yeast cells, the modified cells comprising a genetic alteration that causes the modified cells to produce a decreased amount of functional Dls1 polypeptide compared to the parental cells, wherein the modified cells produce during fermentation (i) an increased amount of alcohol compared to parental cells at the same fermentation temperature and/or (ii) produce the same amount of alcohol compared to the parental cells at a higher fermentation temperature.

2. The modified cells of claim 1, wherein the genetic alteration comprises a disruption of the YJL065c gene present in the parental cells.

3. The modified cells of claim 2, wherein disruption of the YJL065c gene is the result of deletion of all or part of the YJL065c gene.

4. The modified cells of claim 2, wherein disruption of the YJL065c gene is the result of deletion of a portion of genomic DNA comprising the YJL065c gene.

5. The modified cells of claim 2, wherein disruption of the YJL065c gene is the result of mutagenesis of the YJL065c gene.

6. The modified cells of any of claims 2-5, wherein disruption of the YJL065c gene is performed in combination with introducing a gene of interest at the genetic locus of the YJL065c gene.

7. The modified cells of any of claims 1-6, wherein the cells do not produce functional Dls1 polypeptide.

8. The modified cells of any of claims 1-6, wherein the cells do not produce Dls1 polypeptide.

9. The modified cells of any of claims 1-8, wherein the cells further comprise an exogenous gene encoding a carbohydrate processing enzyme.

10. The modified cells of any of claims 1-9, further comprising an alteration in the glycerol pathway and/or the acetyl-CoA pathway.

11. The modified cells of any of claims 1-10, further comprising an alternative pathway for making ethanol.

12. The modified cells of any of claims 1-11, further comprising a pathway for making butanol.

13. The modified cells of any of claims 1-12, wherein the cells are of a Saccharomyces spp.

14. A method for producing a modified yeast cell comprising: introducing a genetic alteration into a parental yeast cell, which genetic alteration reduces or prevents the production of functional Dls1 polypeptide compared to the parental cells, thereby producing modified cells that produces during fermentation (i) an increased amount of alcohol compared to parental cells at the same fermentation temperature and/or (ii) produce the same amount of alcohol compared to the parental cells at a higher fermentation temperature.

15. The method of claim 14, wherein the genetic alteration comprises disrupting the YJL065c gene in the parental cells by genetic manipulation.

16. The method of claim 14 or 15, wherein the genetic alteration comprises deleting the YJL065c gene in the parental cells using genetic manipulation.

17. The method of any of claims 14-16, wherein disruption of the YJL065c gene is performed in combination with introducing a gene of interest at the genetic locus of the YJL065c gene.

18. The method of any of claims 14-17, wherein disruption of the YJL065c gene is performed in combination with making an alteration in the glycerol pathway and/or the acetyl-CoA pathway.

19. The method of any of claims 14-18, wherein disruption of the YJL065c gene is performed in combination with adding an alternative pathway for making ethanol.

20. The method of any of claims 14-19, wherein disruption of the YJL065c gene is performed in combination with adding a pathway for making butanol.

21. The method of any of claims 14-20, wherein disruption of the YJL065c gene is performed in combination with introducing an exogenous gene encoding a carbohydrate processing enzyme.

22. The method of any of claims 14-21, wherein the modified cell is from a Saccharomyces spp.

23. The method of any of claims 14-22, wherein the alcohol is ethanol and/or isobutanol.

24. Modified yeast cells produced by the method of any of claims 14-23.


SEQUENCE LISTING (18 sequences)

SEQ ID NO: 1 - protein, 167 aa, S. cerevisiae
MNNETSGKETASAPLCSPKLPVEKVQRIAKNDPEYMDTSDDAFVATAFATEFFVQVLTHESLHRQQQQQQQQVPPLPDELTLSYDDISAAIVHSSDGHLQFLNDVIPTTKNLRLLVEENRVRYTTSVMPPNEVYSAYVVNDTAPKPNIVEIDLDNDEDDDEDVTDQE

SEQ ID NO: 2 - protein, 167 aa, S. cerevisiae
MNNETSGKETASAPLCSPKLPVEKVQRIAKNDPEYMDTSDDAFVATAFATEFFVQVLTHESLHRQQQQQQQQVPPLPDELTLSYDDISAAIVHSSDGHLQFLNDVIPTTKNLRLLVEENRVRYTTSVMPPNEVYTAYVVNDTAPKPNIVEIDLDNDEDDDEDVTDQE

SEQ ID NO: 3 - protein, 167 aa, S. cerevisiae
MNNETSGKETASAPLCSPKLPVEKVQRIAKNDPEYMDTSDDAFVATAFATEFFVQVLTHESLHRQQQQQQQQVPPLPDELTLSYDDISAAIVHSSDGHLQFLNDVIPTTKNLRLLVEENRVRYTTSVMPPNEVYSAYVVNNTAPKPNIVEIDLDNDEDDDEDVTDQE

SEQ ID NO: 4 - protein, 167 aa, S. cerevisiae
MNNETSGKETASAPLCSPKLPVEKVQRIAKNDPEYMDTSDDAFVATAFATEFFVQVLTHESLHRQQQQQQQQVPPLPDELTLSYDDISAAIVHSSDGHLQFLNDVIPTTKNLRLLVEENRVRYTTSVIPPNEVYSAYVVNDTAPKPNIVEIDLDNDEDDDEDVTDQE

SEQ ID NO: 5 - protein, 167 aa, S. cerevisiae
MNNETSGKETASAPLCSPKLPVEKVQRIAKNDPEYMDTSDDAFVATAFATEFFVQVLTHESLHRQQQQQQQQVPPLPDELTLSYDDISAAIVHSSDGHLQFLNDVIPTTKNLRLLVEENRVRYTTSVMPPNEVYSAYVVNDTAPKPNIVEIDLDNDEDDDDDVTDQE

SEQ ID NO: 6 - protein, 167 aa, S. cerevisiae
MNNETSGKETASAPLCSPKLPVEKVQRIAKNDPEYMDISDDAFVATAFATEFFVQVLTHESLHRQQQQQQQQVPPLPDELTLSYDDISAAIVHSSDGHLQFLNDVIPTTKNLRLLVEENRVRYTTSVMPPNEVYSAYVVNDTAPKPNIVEIDLDNDEDDDEDVTDQE

SEQ ID NO: 7 - protein, 167 aa, S. cerevisiae
  misc_feature (73)..(73): Xaa can be any naturally occurring amino acid
MNNETSGKETASAPLCSPKLPVEKVQRIAKNDPEYMDTSDBAFVATAFATEFFVQVLTHESLHRQQQQQQQQXPPLPDELTLSYDDISAAIVHSSDGHLQFLNDVIPTTKNLRLLVEENRVRYTTSVMPPNEVYSAYVVNDTAPKPNIVEIDLDNDEDDDEDVTDQE

SEQ ID NO: 8 - protein, 167 aa, S. cerevisiae
MNNETSGKETASAPLCSPKLPVEKVQRIAKNDPEYMDTSDDAFVATAFATEFFVQVLTHESLHRQQQQQQQQVPALPDELTLSYDDISAAIVHSSDGHLQFLNDVIPTTKNLRLLVEENRVRYTTSVMPPNEVYSAYVVNDTAPKPNIVEIDLDNDEDDDEDVTDQE

SEQ ID NO: 9 - protein, 167 aa, S. cerevisiae
MNNETSGKETASAPLCSPKLPVEKVQRIAKNDPEYMDTSDDAFVATAFATEFFVQVLTHESLHRQQQQQQQQVPPLSDELTLSYDDISAAIVHSSDGHLQFLNDVIPTTKNLRLLVEENRVRYTTSVMPPNEVYSAYVVNDTAPKPNIVEIDLDNDEDDDEDVTDQE

SEQ ID NO: 10 - protein, 167 aa, S. cerevisiae
MNNETSGKETASAPLCSPKLPVEKVQRIAKNDPEYMDTSDDAFVATAFATEFFVQVLTHESLHRQQQQPQQQVPPLPDELTLSYDDISAAIVHSSDGHLQFLNDVIPTTKNLRLLVEENRVRYTTSVMPPNEVYSAYVVNDTAPKPNIVEIDLDNDEDDDEDVTDQE

SEQ ID NO: 11 - protein, 167 aa, S. cerevisiae
MNNETSGKETASAPLCSPKLPVEKVQRIAKNDPEYMDTSDDAFVATAFATEFFVQVLTHESLHRQQQQQQQQVPPLPDELTLSYDDISAAIVHSSDGHLQFLNDVIPTTKNLRLLVEENRVRYITSVMPPNEVYSAYVVNDTVPKPNIVEIDLDNDEDDDEDVTDQE

SEQ ID NO: 12 - protein, 167 aa, S. cerevisiae
MNNETSGKETASAPLCSPKLPVEKVQRIAKNDPEYMDTSDDAFVATAFATEFFVQVLTHESLHRQQQQQQQQVPALPDELTLSYDDISAAIVHSSDGHLQFLNDVIPTTKNLRLLVEENRVRYTTSVIPPNEVYSAYVVNDTAPKPNIVEIDLDNDEDDDEDVTDQE

SEQ ID NO: 13 - protein, 162 aa, S. kudriavzevii
MSDDTSRIEAASPPPYSLQLPVEKVQRIAKNDPEYMDTSDDAFIATALATESFIQVLALESLQHQVPRQVPHPSDEITLSYDDISGTIVRSADGHLQFLNDVIPMTKNLRLLVEENRVRYTTSVMPPNEVYSGCVMNETASKPDIVEIDLDNDEDEDVTDQE

SEQ ID NO: 14 - protein, 164 aa, S. eubayanus
MNENTSSIETGVPPPCSLQLPVEKVQRIAKNDPEYMDTSDDAFVATAFATEFFIQVLTHESLQQQQRGQVPHPSDEITLSYDDVSATILKSTDGHLQFLNDVIPITKNLRLLVEENRVRYTTSVMPPNEVYSTYVMGETALKPNIVEIDLDNDEDDDEDVTDQE

SEQ ID NO: 15 - protein, 162 aa, S. kudriavzevii
MSDDTSRIDAASPPPYSLPAACGKVQRIAKNDPEYMDTSDDAFIATALATESFIQVLALESLQHQVPRQVPHPPDEITLSYDDISGTIVRSADGHLQFLNDVIPMTKNLRLLVEENRVRYTTSVMPPNEVYSGCVMNETASKPDIVEIDLDNDEDEDVTDQE

SEQ ID NO: 16 - protein, 166 aa, S. arboricola
MENDANGTETVSPPPHSPQLPVEKVQRIAKNDPEYMDTSDDAFVATAFAAQFFIQLLTHESLQQQQQRHHQILHPSDEITLSYDDISATILRSTDGHLQFLNDVIPLTKNLRLLVEENRVRYTTSVVPPNEVYSAYMMNETGVKPNIIEIDLDNDEDDDEDVTDQE

SEQ ID NO: 17 - protein, 167 aa, S. cerevisiae; identical to SEQ ID NO: 4

SEQ ID NO: 18 - DNA, 504 bp, S. cerevisiae
atgaacaacgagactagtggtaaagaaacggcgtctgcacctctgtgttcgcccaagttacctgtagaaaaagtgcagagaatagccaagaatgatccagaatatatggacacttcggatgacgcattcgtagccacagcgtttgctacagaattcttcgtccaggtgctgacacatgagtccctacataggcaacagcagcagcaacaacaacaggtaccgccgctcccagatgaactcacgctgtcgtacgatgacatctctgccgcaattgtgcactcttctgacggccatctgcagtttttgaatgatgtgataccaacaacaaagaatttgaggcttctagtggaagaaaaccgagttagatatactacaagtgtcatgccccctaatgaagtttactccgcctatgtggtgaacgatacggctccgaagcccaacattgtcgagattgatcttgataatgacgaagacgacgacgaagacgttactgatcaagaataa